(19) United States
(12) Patent Application Publication
(10) Pub. No.: US 2024/0378826 A1
(43) Pub. Date: Nov. 14, 2024

(54) FAST, DYNAMIC REGISTRATION WITH AUGMENTED REALITY

(71) Applicant: Monogram Orthopaedics Inc., Austin, TX (US)

(72) Inventors: Kamran SHAMAEI, Austin, TX (US); Pedro Alfonso PATLAN ROSALES, Austin, TX (US); Hrisheekesh PATIL, Austin, TX (US)

(73) Assignee: Monogram Orthopaedics Inc., Austin, TX (US)

(21) Appl. No.: 18/763,090

(22) Filed: Jul. 3, 2024

Related U.S. Application Data
(63) Continuation of application No. PCT/US2023/060029, filed on Jan. 3, 2023.
(60) Provisional application No. 63/266,380, filed on Jan. 4, 2022.

(51) Int. Cl. G06T 19/00; A61B 34/10; G06T 7/33; G16H 20/40

(52) U.S. Cl. CPC G06T 19/006; A61B 34/10; G06T 7/344; G16H 20/40; A61B 2034/105; G06T 2207/20101; G06T 2210/41

(57) ABSTRACT

Fast, dynamic registration with augmented reality includes registering a model point cloud to a point cloud of an object, including obtaining selection of an origin point for the model point cloud as a sampled surface point on the object in an established collection of sample points of the object, the collection forming the point cloud of the object; obtaining other sampled surface point(s) on the object and including those in the collection; determining an initial pose of the model point cloud based on the collection of sample points; obtaining an additional sampled surface point and updating the collection to include such; determining a fit of the model point cloud to the point cloud of the object based on the updated collection; determining a registration accuracy of the fit of the model point cloud to the point cloud of the object; and performing processing based on the determined registration accuracy.

### BACKGROUND

Registration in medical imaging refers to processes for finding the relationship between one coordinate frame/system and another coordinate frame/system. This relationship is termed a ‘transformation’. In many applications of registration, the two point clouds represent the same physical body, and the registration is to align the point cloud in one coordinate frame to the point cloud in the other coordinate frame. In medical imaging applications, there may be an anatomical model, for instance, a model of a bone of a patient, that is presented in one coordinate frame, and that is to be registered to the actual anatomy of the patient in another coordinate frame. The anatomical model of the patient anatomy is often produced by way of a computed tomography (CT) scan or other diagnostic imaging technique and presents features of the patient anatomy (e.g., bone) for operative/surgical planning against that model. During the operative procedure involving the patient anatomy, the model is to be registered to the image/view of the patient anatomy that the model represents. ‘Arrays’ may be rigidly fixed to the patient, for instance to the patient bone, to serve as trackable markers for imaging systems that can then be used to ascertain a transform to know the exact location of the patient anatomy and features in space. A registration probe may be used to make surface contact with the anatomy (e.g., bone) and assign position coordinates to the probe tip at each such registered point to produce a surface point cloud of the patient anatomy. The model can then be registered to those features.

### SUMMARY

Shortcomings of the prior art are overcome and additional advantages are provided through the provision of a computer-implemented method. The method includes registering a model point cloud to a point cloud of an object. The registering includes obtaining a user selection of an origin point for the model point cloud, the origin point being a sampled surface point on the object and being a first point included in an established collection of sample points of the object, the collection forming the point cloud of the object, obtaining one or more other sampled surface points on the object and including the obtained one or more other sampled surface points in the collection, determining an initial pose of the model point cloud based on the collection of sample points of the object, obtaining an additional sampled surface point on the object and updating the collection of sample points to include the additional sampled surface point and thereby provide an updated collection of sample points, determining a fit of the model point cloud to the point cloud of the object based on the updated collection of sample points of the object, determining a registration accuracy of the fit of the model point cloud to the point cloud of the object, and performing processing based on the determined registration accuracy.

Further, a computer system is provided that includes a memory and a processor in communication with the memory, wherein the computer system is configured to perform a method. The method includes registering a model point cloud to a point cloud of an object. The registering includes obtaining a user selection of an origin point for the model point cloud, the origin point being a sampled surface point on the object and being a first point included in an established collection of sample points of the object, the collection forming the point cloud of the object, obtaining one or more other sampled surface points on the object and including the obtained one or more other sampled surface points in the collection, determining an initial pose of the model point cloud based on the collection of sample points of the object, obtaining an additional sampled surface point on the object and updating the collection of sample points to include the additional sampled surface point and thereby provide an updated collection of sample points, determining a fit of the model point cloud to the point cloud of the object based on the updated collection of sample points of the object, determining a registration accuracy of the fit of the model point cloud to the point cloud of the object, and performing processing based on the determined registration accuracy.

Yet further, a computer program product including a computer readable storage medium readable by a processing circuit and storing instructions for execution by the processing circuit is provided for performing a method. The method includes registering a model point cloud to a point cloud of an object. The registering includes obtaining a user selection of an origin point for the model point cloud, the origin point being a sampled surface point on the object and being a first point included in an established collection of sample points of the object, the collection forming the point cloud of the object, obtaining one or more other sampled surface points on the object and including the obtained one or more other sampled surface points in the collection, determining an initial pose of the model point cloud based on the collection of sample points of the object, obtaining an additional sampled surface point on the object and updating the collection of sample points to include the additional sampled surface point and thereby provide an updated collection of sample points, determining a fit of the model point cloud to the point cloud of the object based on the updated collection of sample points of the object, determining a registration accuracy of the fit of the model point cloud to the point cloud of the object, and performing processing based on the determined registration accuracy.

In embodiments, the model point cloud comprises an anatomy model point cloud and the object comprises a patient anatomy.

In embodiments, the performing processing includes, based on the determined registration accuracy being less than a preconfigured threshold level of accuracy, iterating, one or more times, the obtaining an additional sampled surface point, the determining a fit, and the determining the registration accuracy. In embodiments, the iterating halts based on the determined registration accuracy being at least the preconfigured threshold level of accuracy. In embodiments, based on halting the iterating, the determined fit of the bone model point cloud to the point cloud of the patient anatomy provides a registration of the bone model point cloud to the point cloud of the patient anatomy, and the method further includes determining and digitally presenting to a surgeon one or more indications of surgical guidance.

In some embodiments, obtaining the user selection of the origin point includes providing a bone model augmented reality (AR) element overlaying a portion of a view to the patient anatomy. The view can show a registration probe, and the bone model AR element can be provided at a fixed position relative to a probe tip of the probe. User movement of the probe can reposition the bone model AR element and the user selection of the origin point can include the user positioning and orienting the bone model AR element in the view to overlay the patient anatomy by touching the patient anatomy with the probe tip, and then providing some input (e.g., a mouse click, button press, verbal confirmation, or the like) to select the origin point as a position of the probe tip touching the patient anatomy. Further, in some examples obtaining the user selection of the origin point includes providing a probe axis AR element overlaying another portion of the view to the patient anatomy. The probe axis AR element can include an axis line extending from the probe at a first position (for instance the tip) and away from the probe tip to a second position, where the axis line represent an axis of the probe/probe tip.

Additionally or alternatively, determining the fit of the bone model point cloud to the point cloud of the patient anatomy based on the updated collection of sample points of the patient anatomy can include performing a rough fitting of the bone model point cloud to the point cloud of the patient anatomy using the updated collection of sample points of the patient anatomy and, based on performing the rough fitting, performing a fine fitting of the bone model point cloud to the point cloud of the patient anatomy using the updated collection of sample points of the patient anatomy. In embodiments, performing the rough fitting includes applying a random sample consensus (RANSAC) algorithm and/or performing the fine fitting includes applying an iterative closest point (ICP) algorithm.

Additionally or alternatively, determining the initial pose of the bone model point cloud can also utilize rough-fitting and/or fine-fitting. For instance, determining the initial pose of the bone model (e.g., after the first two or three sampled points for instance) can include performing a rough fitting of the bone model point cloud to the point cloud of the patient anatomy by applying a random sample consensus (RANSAC) algorithm and, based on performing the rough fitting, performing a fine fitting of the bone model point cloud to the point cloud of the patient anatomy by applying an iterative closest point (ICP) algorithm.

Additional features and advantages are realized through the concepts described herein.

### BRIEF DESCRIPTION OF THE DRAWINGS

Aspects described herein are particularly pointed out and may be distinctly claimed, and objects, features, and advantages of the disclosure are apparent from the detailed description herein taken in conjunction with the accompanying drawings in which:

FIG. 1 depicts an example environment for point sampling against patient anatomy in accordance with aspects described herein;

FIGS. 2 and 3 depict examples of AR-assisted bone model origin selection by a user and model positioning relative to patient anatomy in accordance with aspects described herein;

FIG. 4 depicts an example of solving for a local minimum to infer an impossible solution in fitting a model to patient anatomy;

FIG. 5 depicts an example process for fitting the bone model point cloud to a surface point cloud of patient anatomy, in accordance with aspects described herein;

FIG. 6 depicts an example visualization of updating a registration transform for a bone model point cloud based on additional sampled points, in accordance with aspects described herein;

FIG. 7 depicts an example of AR-assisted sample point identification in accordance with aspects described herein;

FIG. 8 depicts one example of a computer system and associated devices to incorporate and/or use aspects described herein

FIG. 9 depicts one example of a smart eyewear device;

FIG. 10 depicts an example limitation of not rendering tracking arrays as virtual objects; and

FIG. 11 depicts how orientation of the registration probe need not affect the coordinates of the sampled point.

### DETAILED DESCRIPTION

There are drawbacks to existing approaches for registration in surgical planning and other applications. For instance, they often require a relatively large number (e.g., 40 or more) of sampled points per bone using the probe. Additionally, they often require that points be sampled in a specified order, requiring the surgeon to follow a guidance application with on-screen prompts to sample specific points indicated by the system. Furthermore, the existing registration workflows necessitate that all of the points be sampled to advance to the next steps in the workflow, irrespective of whether they are required or actually improve the registration accuracy. By way of specific example in which a surgical procedure is performed on a knee, particular systems require sampling of 40 points on each of the femur and tibia (80 points total) in order to advance to the next steps in the workflow. Additionally, some require sampling of points not contiguous with many of the other collected points, which is disruptive to workflow. For example, some systems require point samples on the distal end of the femur, proximal points on the femur, as well as the inner and outer malleolus, which is a different anatomy.

Many current registration algorithms rely on Iterative Closest Point (“ICP”) calculations to determine the transform of the tracked object to its pre-operative reference frame (which could be an approximation, for example, with imageless systems). ICP algorithms seek to minimize the differences between two clouds of points. It is possible for ICP algorithms to output results that do not represent the minimal difference between the point clouds (i.e., local minima).

In addition to the above (and using the example of a knee procedure), some approaches require manipulations of the leg, in which the leg must be removed from the knee positioner, manipulated, and then re-constrained, to register the hip center.

Many robot and navigation companies use infrared (IR)-based tracking cameras that require 3D model renderings of the surgical theater, including the patient anatomy and surgical instruments. The rendering of these objects can be glitchy and often does not include a backdrop for context (i.e., the objects appear to be floating in space on a blank screen). These issues involving existing digital representations can produce frustration and divert the surgeon's attention away from the surgery to focus on screens with imperfect renderings of the surgery.

Rendered objects often have artifacts, latency, and other drawbacks that result in an inaccurate depiction of reality. With registration, such latency and errors can cause frustration during point sampling, for instance if a surgeon is required to touch the tip of a rendered probe to a specific point on a rendered bone model. Latency in updating the model, for instance after removal of a portion of the bone, can cause additional frustrations. A probe tip may show that it has penetrated the bone (which is highly unlikely) when it has not.

Many surgeons do not understand the purpose of registration, which can also lead to frustration and disengagement. Some existing systems do not provide visual cues to help the surgeon understand the purpose of their actions or to help provide an error check on registration accuracy. Additionally, current systems do not include fail-safes against collecting points without any penalty before registration. For instance, a surgeon might introduce error by unintentionally collecting a point in the air (without touching the bone).

Accordingly, current approaches suffer from one or more of these and/or other drawbacks by increasing surgical time, requiring additional, and frustrating, steps for the user (e.g., ordered points vs. randomly sampled points), and/or requiring sampling of incongruous points (e.g., hip center and medial and lateral malleolus), while likely being less accurate and prone to general disengagement of the user from thoughtful participation in the registration workflow.

Described herein are approaches that enable faster registration with minimized disruption to operative workflows and without compromising accuracy. Aspects propose the use of reality augments, improved algorithms, and thoughtful point sampling to reduce sampling time and provide a user-friendly workflow. As noted, registration may commonly be used in navigation systems, for example, robotics. While examples described herein are presented in the context of registration between a bone model and actual patient anatomy, i.e., the point clouds of each, for use in conjunction with surgical procedure guidance, aspects of registration approaches described herein are more widely applicable outside of anatomical registrations and surgical applications.

As noted, a common registration process calculates a coordinate frame of a rigidly mounted trackable array, typically one array per bone, relative to a bone coordinate frame through a process of point sampling with a tracked registration probe. A point cloud is generated using a registration probe to make bone surface contact and assign position coordinates to the tip at each registered point. Thus, the point cloud represents a set of data points in space that correspond to the surface anatomy of the patient's bone.

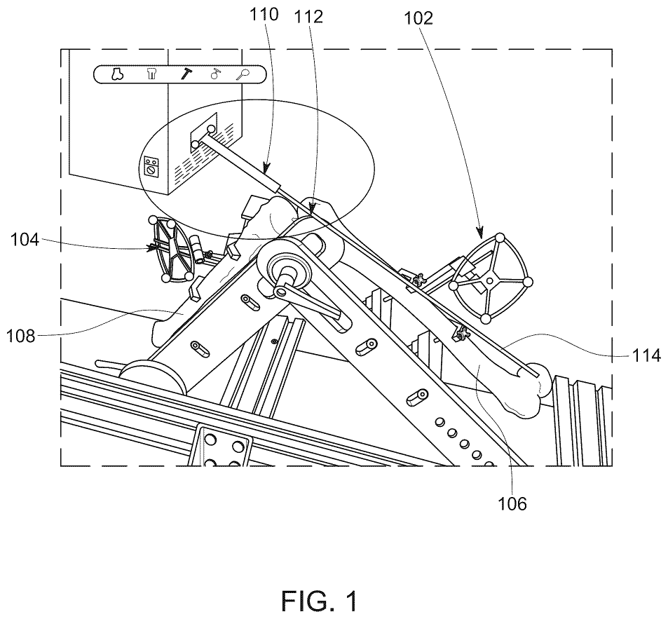

Referring to FIG. 1, an example registration determines the position of fixed tracking arrays (102, 104) coupled to patient anatomy (femur 106 and tibia 108—the fibula is not depicted in FIG. 1). The position of an array may be found via point sampling of points on the bone surface, sampling the medial and lateral malleolus, and inferring hip center with leg manipulation. A probe 110 having a probe tip 112 samples a point at the end of the femur 106 in this example.

As described above, some existing approaches require a large number (e.g., 40) of point samples on each of the distal end of the femur and proximal end of the tibia, and additional samples from the medial and lateral points of the malleoli of the tibia 108. This can be cumbersome, as noted.

FIG. 1 also depicts a line 114 as a central axis line representing an axis of the probe 110, and extending from the probe 110 (from the probe tip 112 in this example) to the end of the femur in this example. In accordance with aspect described herein, this is a guide line and may be presented for the surgeon as an augmented reality (AR) element. As explained in further detail below, the surgeon can orient the probe such that this line extends as close as possible through the central axis of the bone (e.g., femur 106) to point the line at the patient's hip center. This can be utilized in place of a physical manipulation of the leg to orient the bone model such that the bone model and actual patient bone are relatively closely coaxially aligned.

In examples, a human (such as the surgeon) is involved in performing the point sampling using the probe to register the bone model to the patient anatomy. An operative procedure performed based on the registration, for instance to cut bone, insert medical devices, etc., could be performed by surgeon(s), robot(s) (with or without human involvement), or a combination of the two. Notably, pre-operative data, for example a CT scan, may contain more information than is visible to the surgeon during the procedure. For example, a CT scan captures the thickness of the cortical wall. Accurate registration correlates this preoperative data to the real-time pose of the anatomy so that the surgeon has access to additional patient information. Thus, based on the registration, a process could, for instance, determine and digitally present, to a surgeon, and relative to the actual patient anatomy, one or more indications of surgical guidance determined based on the bone model.

Registration methods provided herein may be faster, more accurate, and easier to perform. This may be done without requiring, e.g., ordered points or leg manipulations to infer the hip center or samples of the medial or lateral malleolus. They may be easier to use because of innovative reality augments that help the accuracy of bone model pose and point sampling as described herein. Meanwhile, registration accuracy may be checked during point sample collection rather than waiting to the end of sample collection. This can be used to determine when registration is complete (e.g., the registration accuracy based on the latest sampled point meets a desired threshold), and thereby avoid the user having to sample additional points when they are not needed to achieve the desired level of registration accuracy. Additionally, aspects engage the user in the registration workflow through visual cues. “User” as used herein refers to the user using a system to proceed through a registration process. Often, this will be the surgeon and therefore the term “user” and “surgeon” may be used interchangeably herein, though it is noted that the user collecting the sample points need not necessarily be the surgeon and could instead be an assisting medical practitioner, for instance.

Registration methods provided herein may also reduce the occurrence of failed registrations, i.e., registrations for which the minimum accuracy threshold conditions are not achieved. Failed registrations are problematic because they introduce surgical time and user frustration.

These and other aspects can be helpful for any navigated surgical procedure, not just those discussed or depicted herein involving a knee but also spine and other anatomies. Additionally, aspects may apply in other industrial and/or navigated applications to register point clouds.

In accordance with some aspects, visible imaging sensor(s), e.g., red, green, blue wavelength (RGB) camera(s), is/are used. An RGB camera provides a view of the environment/surgical theater for a human (e.g., the surgeon) to understand the environment. Such camera(s) may be used together with a tracking system that tracks patient anatomy in space. An infrared (IR)-based tracker may serve as such a tracking system, though there are other example facilities/algorithms that might be used.

By way of specific example, a Polaris Vega® VT optical tracker offered by Northern Digital Inc., Waterloo, Ontario, Canada (of which VEGA is a registered trademark) may be utilized, which encompasses an integrated high definition video camera and IR camera(s). In the noted VT system, the IR data coordinate system may be aligned to the camera stream.

AR overlays (i.e., as digital elements presented to overlay an image/camera feed) may be provided as explained elsewhere herein.

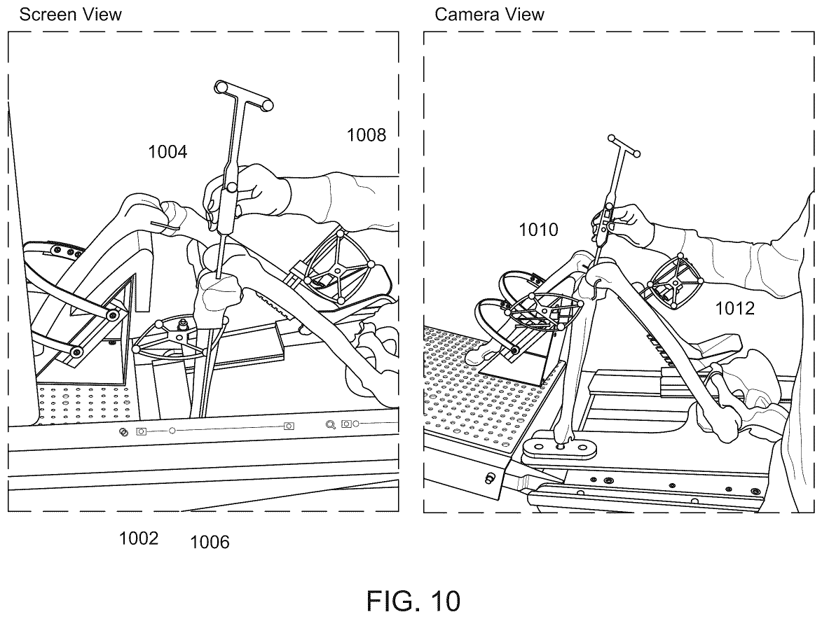

One aspect of approaches discussed herein is to set the origin of the bone model scan (from the CT scan as one example) to a position relative to the corresponding patient anatomy that is easy and intuitive to sample and from which as much helpful information as possible can be inferred. The origin point of the model can define its coordinate system and determine where the object is located in real space. We note that other objects of interest, such as the rigid tracking arrays, may be rendered as virtual reality augments to enhance the dimensionality of the image from the camera frame (to ensure, for example, that objects that are closer to the camera than others do not appear to be behind such objects and visa-versa). Various surgical instruments or objects (such as trackable array(s)) may be rendered as virtual objects to enhance on-screen visualization of camera views. By way of non-limiting example, it may be of interest to render the fixed, rigid arrays (e.g., 104, 104 of FIG. 1) as augmented reality overlays to assist the surgeon with spatial orientation. See FIG. 10 demonstrating a limitation of not rendering the arrays as virtual objects. The tracker array 1002, which is physically closer to the camera than the registration probe 1004 and bone 1006, appears to be behind these objects in the reality augment (Screen View) because it has not been rendered as a virtual reality augment. It may be of interest to render objects of interest, such as the tracking arrays, e.g., tracking arrays 1002 and 1008, as a virtual objects (e.g., as 1010, 1012 in the Camera View) to avoid this concern.

Registration is facilitated, expedited, and ensured accuracy by allowing the user to quickly select the model origin and position relative to the actual patient bone position with an easily-chosen, single sampled starting point aligned with the help of on-screen reality augments. The origin of the bone model is made a useful point because it helps with the initial alignment of the model to the patient anatomy. With a good initial alignment, fewer additional points are needed to accurately and adequately determine the transformation to register the bone model point cloud to the patient anatomy point cloud defined by the sampled points. With respect to the origin point, it may generally be desired that the patient anatomy that corresponds to the bone model origin is easy to access and located such that the axis of the probe tip can intuitively be aligned with the axis of the bone.

By way of non-limiting example, the bone model origin may be a proximal surface point within a cylinder approximated by the bone shaft and generally aligned with the tubercle of the bone. Approximating the bone as a cylinder, it may be beneficial to set the bone model origin to a surface point inside the cylinder. For instance, the bone origin may be selected to be a distal point (femur) or proximal point (tibia) that runs through an approximated axis of the bone. In the example of FIG. 1, the origin may be set at the point on the surface of the femur 106 at the tip 112 of the probe 110. We note that the origin may be any point for which initial placement and orientation of the probe with respect to the anatomy and an AR overlay is intuitive. By way of non-limiting example, the origin may be a point on the distal surface or the femur 106 or proximal surface of the tibia 108. While it is generally most acceptable for the probe tip to contact bone, and thus for sample points to be intra-incisional, we note that the probe could sample the bone surface through the skin.

A system in accordance with aspects described herein can automatically help a user choose the best initial point/alignment. For instance, the bone model can be presented to the user in AR overlay that displays the patient anatomy in a fixed position relative to the probe tip. The user can manipulate the probe to orient and position the bone model to coincide with the patient's anatomy, i.e., visually fit the model to the appropriate position.

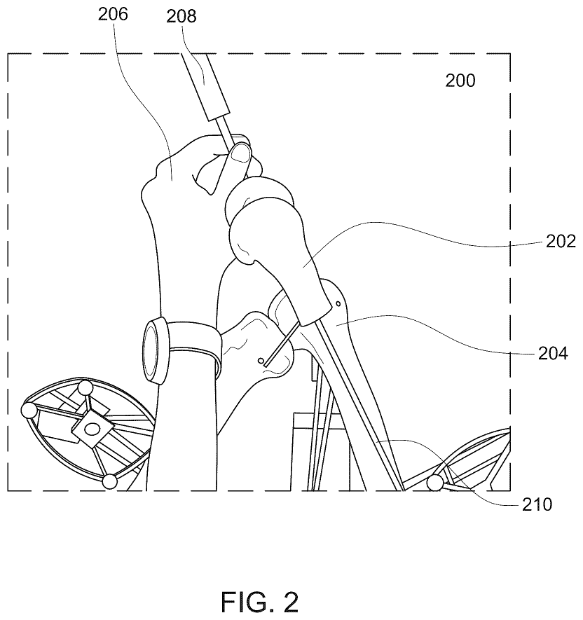

Referring to FIG. 2, shown is a bone model 202 presented as an AR element imposed over a view of an environment 200 that includes a patient bone 204. For instance, the view may be provided by a camera feed, and a computer system can impose AR elements over the view and display the view with AR elements on a screen. Additionally or alternatively, the user could wear smart glasses or other wearable devices to view the environment through a transparent display(s) (such as transparent lenses with active displays built therein), and the AR element(s) could be presented on the transparent display to provide the augmented view for the user. Shown also in FIG. 2 is the user's arm/hand 206 holding probe 208, specifically a shaft of the probe. At the end of this shaft is the probe tip (just below the user's thumb in FIG. 2). Since the exact location of the probe tip is known by way of probe tracking provided with the probe, the system can place the AR bone model origin at the tip of probe 208, as shown in FIG. 2. As the uses lowers the probe in this view, the bone model 202 travels with the probe, remaining in the fixed position and orientation relative to the probe tip. The model ‘floats’ and moves around with the probe tip. If the user reorients the probe to change the axis of the probe tip (indicated by the line 210), then the axis of the bone model will change accordingly. Here, the shaft of bone 204 is approximated to a cylinder and the line 210 (also an AR element) is provided to represent the probe axis, which can be visually aligned to correspond to a bone axis. The user can align the line 210 with the axis of the patient's bone 204 as visually estimated by the user.

In this manner, the user holds the probe, moving and twisting it to orient (in position and rotation) the bone model 202 to the specific object of interest—the upper portion of the femur 204 in this example. Since the bone model 202 originates from the tip of the probe 208, it is expected that the probe tip will touch a surface of the patient's bone when the model is in an approximately correct position and orientation. The user can then provide some input (keystroke, mouse click, button press on the probe, etc.) to select the origin point and temporarily lock-in the position of the bone model originating from that point. With this user selection, the initial alignment of the bone model 202 is selected and the model is placed in that position (i.e., as reflected in AR) that the user selected. From there, the user can move the probe 208 to collect other sample points on the patient anatomy as described below. As the user samples additional points, this provides the system with additional actual bone surface points, taken as truths of the location of the bone surface. Each additional truth can result in the system slightly adjusting the position of the model to fit the model to the points collected to that point in the process. The registration of the bone model is expected to become more accurate with each additional point sampled. We note that all of the captured data points may be processed either simultaneously or in parallel by algorithms that help with pose determination. By way of nonlimiting example, an outlier detection algorithm (for example, a Random Sample Concensus algorithm) and an Iterative Closest Point algorithm (for example, an ICP algorithm) may take all sampled points of interest as data inputs and process such data points in parallel or in series to determine the relevant registration transform.

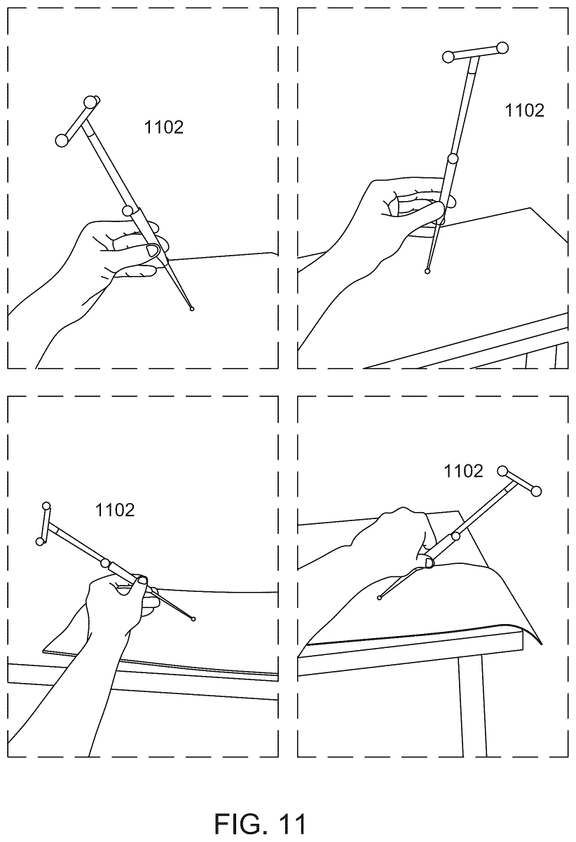

In conventional systems, the orientation of the probe when a point is registered is generally not considered to be relevant data, and the goal is merely to capture the coordinates of the probe tip. Registering surface points requires knowing the probe orientation, however, when the point is sampled, the probe position itself is generally thought to be arbitrary and irrelevant (see FIG. 11 depicting how the orientation of the registration probe 1102 in the four depicted scenarios need not affect the coordinates of the sampled point—the position of the probe tip relative to the bone surface is generally the only relevant data input). That is, the surgeon orients the point however practical.

In contrast, aspects described herein assign relevance to the probe orientation, at least for the first sampled point, e.g., the origin point, to provide an initial starting point for a global, rough fitting of the model and fine fitting of the model. The global, rough fitting may be done using sampled points by applying thereto an algorithm to estimate parameters of a model by generally random sampling of observed data, for example Random sample consensus (RANSAC), Maximum Likelihood Estimate Sample Consensus, Maximum A Posterior Sample Consensus, Causal Inference of the State of a Dynamical System, Resampling, HOP Diffusion Monte Carlo, Hough Tranforms, or similar algorithms. By way of nonlimiting example, a “Random sample consensus” (RANSAC) algorithm and the fine fitting may be done by applying thereto a point-to-plane “Iterative closest point” (ICP) algorithm. Rather than simply capturing the coordinates of the origin surface point, aspects establish the coordinates of this point based on the probe's orientation (i.e., the ‘pose’). Because this first point may be taken as the bone model origin, the process properly aligns the origin coordinate frame with the first sampled point. Positioning the origin coordinate frame of the model with the first sampled point can significantly reduce the error metric and the chances of iterating to a local minimum rather than an absolute minimum. In other words, the initial pose provided by the user-selected orientation as explained above enables the system to initially filter some of the infinite possibilities that a fine fitting (e.g., ICP) provides and instead establish a most informative starting point from which initial guesses may be made. The fitting algorithm(s) are provided a general orientation of the model because it is provided relative to the orientation of the probe, which is known. The fine-fit algorithm (e.g., ICP) might otherwise assume that the bone could be anywhere. By providing this initial orientation, it eliminates potentially several ‘local minimums’ that the fine-fit algorithm might otherwise consider to be candidates for orientation. In effect, the initial orientation injects some intelligence into the fitting algorithm with this initial pose; instead of simply creating a surface map and letting an algorithm (e.g., ICP) iteratively solve for a minimum error between the two point clouds (model and patient anatomy), the user defines an approximated initial orientation of the model to eliminate what might otherwise be possible (incorrect) outcomes of the fitting.

To enhance the usefulness of the probe's orientation as a relevant input, aspects use reality augments to help the user properly orient the bone model point cloud to the patient anatomy and make this process intuitive for the use, as shown for instance in FIG. 2. In some examples, the user views a live video stream from camera(s) capturing images of the environment in which the patient anatomy is positioned, and AR element(s) are displayed along with the video stream on display device(s). In other examples, the user's view to the environment is through AR glasses worn by the user and having transparent display(s), e.g., provided as lenses of the glasses. The AR element(s) can be displayed on the transparent display(s) to impose the elements in the user's line of sight through the lenses to the environment.

One example of a reality augment is an AR element of the bone model from a CT scan, though it should be appreciated that aspects would work for imageless systems that do not use advanced imaging. The IR camera(s) or other tracking system can track the registration stylus/probe's real-time position to determine the corresponding movements of the AR overlays so that they move with the probe to enable the positioning shown in FIG. 2. The AR overlays do not need to be patient-specific but may be generalized shapes of interest.

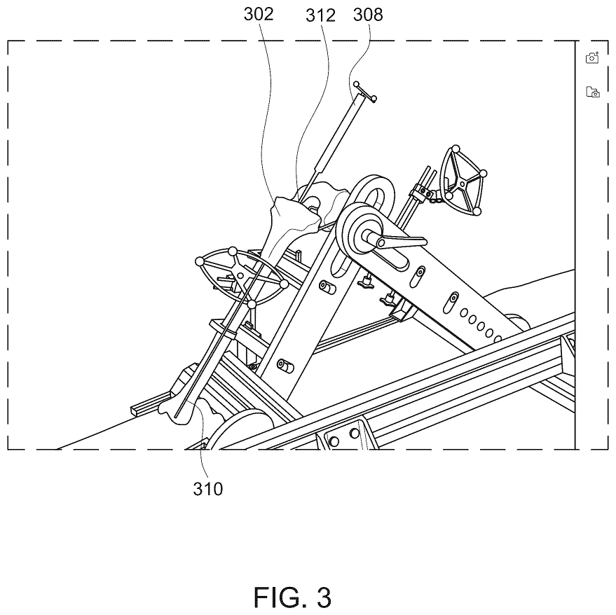

As shown in FIG. 2 and with additional reference to FIG. 3, the bone model 302 can be a section (or optionally the entirety) of the patient's anatomical feature—the bone in this example—displayed at the tip 312 of probe 308 such that the origin of the bone model is the point at the probe tip 312. The transparency of the bone model overlay 302 may be adjusted for usability. The bone model overlay 302 can be rendered such that when the probe tip 312 is placed on the patient's bone surface anatomy to define an origin, the augmented reality overlay will generally be aligned to and overlay the patient's anatomy. In practice, this is immediately intuitive; the user positions the probe to make the AR overlay 302 and patient anatomy at least visually coincident.

FIG. 3 shows AR augments that enhance the usefulness of the registration probe to enable more precise initial positioning of the bone model. The probe tip 312 corresponds to the origin point of the bone model and a virtual line 310 corresponds to a central axis of the probe to assist the user in understanding the probe's orientation. The user can visually align the line 310 to the axis of the patient's bone to assist the user in aligning the bone model to the patient anatomy-beyond what just the bone model itself provides visually since, in this example, the model 302 represents just a portion of the bone. By way of non-limiting example, the line 310 through the axis of the probe tip may be a length extending from the origin to a distal (tibia) or proximal (femur) point that is generally parallel to the bone axis. Notably, this line could be the length from the origin to the hip center for the femur, as that exact length be determined from the initial imaging on which the bone model is based. The line could help with the proper orientation of the probe for the initial sampled point. It is noted that other AR overlays are possible and could be provided to aid the user in positioning the model for the initial sample point/origin.

The AR bone model 302 is placed at the probe tip in these examples but it could be placed anywhere enabling the user to intuitively and easily sample a point on the anatomy surface. It may be generally desired that initial pose selection by the user be intuitive enough so that the user can manipulate the probe to orient the model approximately correctly on the bone. As noted above, the origin may be a root point to which the other sampled points may be referenced, and this origin could be anywhere, though typically it would be on an exposed surface of exposed anatomy (e.g., the top of a bone exposed during surgery) to enable the user to touch the probe tip directly to the surface point on the patient anatomy.

One approach for registration uses, at least in part, iterative closest point (ICP) algorithm(s) for registration. ICP algorithms seek to minimize differences between point clouds. In examples discussed herein, one point cloud is generated by capturing actual bone surface points with the registration probe and the other point cloud corresponds to the bone model generated, for example, by a CT scan. Through a process of trial and error, the algorithm iteratively tries to orient one point cloud to another. The registration accuracy describing how well the position of the bone model point cloud (after it has been transformed) describes the position of the actual patient anatomy can be inferred mathematically. By way of non-limiting example, the registration accuracy could be calculated as the square root of the mean squares of the differences between matched pairs. An ICP algorithm iteratively revises the transformation (the bone model point cloud) to minimize this error metric.

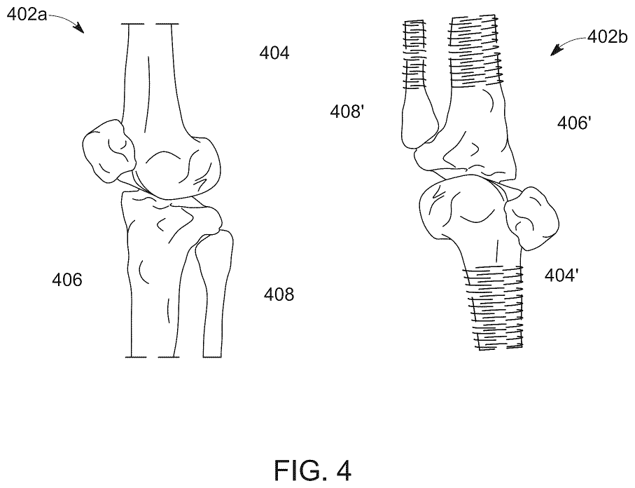

Some conventional anatomical model registrations use an ICP algorithm but notably it lacks “intelligence” in that it iteratively checks the error of transforms that may be random. Because there are infinite possible transforms of a point cloud and the algorithm can only check a finite number of options, a limitation of the ICP algorithm is that it may iteratively solve for a local minimum that is not the absolute minimum. Solving for a local minimum might infer an impossible solution. Referring to FIG. 4, 402a shows the actual patient anatomy (femur 404 above the fibula 408 and tibia 406). The ICP algorithm might minimize the point cloud differences with the model inverted, shown by 402b (with femur 404′, fibula 408′ and tibia 406′), in the solution set of iterations. This illustrates a limitation of an ICP algorithm. In practice, it is common to find a local minimum that is not the actual minimum, producing an orientation that is practically impossible or at the least incorrect. Until a sufficient number of points have been sampled, the results of the ICP algorithm may be very poor and/or nonsensical. Conventional approaches overcome this by increasing the number and diversity of points in the sampled point cloud which, as described above, has drawbacks including increased time spent.

Additionally, current approaches do not incorporate registration accuracy as a real-time variable in registration workflows. Existing systems have a registration protocol that must be followed in its entirety. Only after fully complete does the system calculate the registration accuracy to determine if it falls above or below some defined threshold (for example, 0.5 mm). By way of non-limiting example in a registration protocol calling for 40 sampled points, the registration error may actually be below an allowable registration error threshold after just ten sampled points are collected but this is not known as the user is still required to unnecessarily sample the remaining 30 points before registration and accuracy determination are performed. Furthermore, the user has no sense of the registration error when a point is sampled—the user samples points often without understanding why, and the user has no intuitive way to assess the registration accuracy when progressing through sampling. After the model is fit to the collected points, often the user is presented with data that is not intuitive in the particular application; a surgeon typically would not know the significance or acceptability of a 0.5 mm RMS error, for instance.

In accordance with registration approaches discussed herein, reality augments are used to facilitate a proper orientation of the bone model point cloud to the patient anatomy with the first sampled point by the user as the origin, and multiple fitting algorithms are used. As an example, one fitting algorithm is applied for rough-fitting the orientation of the model and another (different) fitting algorithm is applied for fine-fitting the point clouds based on additional sample points. The RANSAC algorithm, as an example rough fitting, may be used in parallel or in series to the ICP algorithm for outlier detection and to help to find an initial pose for a preliminary transformation, while the RANSAC (or a similar algorithm to estimate parameters of a model by generally random sampling of observed data) and/or ICP may be used for refinement of the transformation. Notably, the two algorithms can be run simultaneously or in sequence.

As described above with reference to FIGS. 2 and 3, the user moves the probe tip into the field of view and selects an initial placement of the bone model point cloud to select an origin point and inform an initial transformation. The user's identification of the origin point in this manner provides the first sampled point of the point cloud of the patient anatomy. The user then samples another one or more points on the patient anatomy with the probe. These one or more points may be selected arbitrarily by the user or based on point(s) suggested by the system. At some point after initial placement and sampling the additional one or more points, the rough (or “global”) fit (e.g., RANSAC) algorithm is applied. In general, the global fit provides a rough alignment/fitting by searching in a relatively large area around the sampled points. In the event that the initial placement of the bone model is relatively far away from the patient anatomy, the global fit provides a better initial alignment for that bone model. The global fit at this point can provide an adjustment to the user's initial fitting. After this rough-fit, a fine-fit, such as one applying ICP, is performed to provide a more focused fit of the bone model to the points that were sampled to that point. We note that the global fitting algorithm may be run at the same time as the ICP. The ICP fit may be performed on the output of the rough-fitting. After this fine-fit, registration accuracy (for instance the error metric) is determined. Assuming the registration accuracy is less than some configurable threshold, then point sampling can continue with global and/or fine fitting performed after each additional sample point(s) is/are collected.

By way of specific example, the process obtains the origin point of the initial pose selected by the user, then obtains one or two additional user samples of the patient anatomy for a total of two or three points constituting the patient anatomy point cloud. At that point the RANSAC algorithm is applied to produce a rough fit, then the ICP algorithm is applied for a finer fit. A determination is made as to whether registration is sufficiently accurate. Depending on how accuracy is measured, the threshold may be a maximum or a minimum threshold. If accuracy is expressed by way of an error measurement (such as in the RMS method), then the threshold may be a maximum allowable error, for instance 0.5 mm or ‘less than 0.5 mm’. Assuming the registration is not to the desired accurate at that point, then the process obtains another (i.e., one additional) point sample of the patient anatomy. The user samples the anatomy surface using the probe and a fit is again performed, this time using the additional sampled point. The fit can again include a rough fit using all the collected points followed by a fine fit using all of the collected points, or may include just one such fit (for instance the fine fit). The registration accuracy may again be determined and the process can proceed either by iterating (if accuracy is below what is desired) or halting if the desired accuracy is achieved. In this manner, the process can iterate through point collection, fitting, and accuracy determination until the registration accuracy is sufficient.

In some examples, the rough and fine fittings are performed after each additional sampled point until a threshold precision in the fit is reached. In other examples, more than one additional point is collected in an iteration before performing the refitting for that iteration.

In this manner, a global fit (e.g., RANSAC) and a fine fit (e.g., ICP) may be performed using sampled points of the patient anatomy and applied as point sampling progresses, e.g., between the sampling of the points.

In some examples, the global fit is performed once after the first n points are collected (n>=3), and then only the fine fit is applied after that, for instance after each additional point is sampled. In other examples, the global fit and fine fit are used as described above after each additional point is sampled. In yet other examples, the global fit may be applied periodically or aperiodically during sampling, for instance after every k number of additional samples are collected, with fine fitting optionally performed after each sample is collected. The iterating through sample collection, fitting, and accuracy determination can stop and end once the accuracy determination determines that the desired accuracy in the registration of the point clouds has been achieved.

In some examples, registration accuracy is determined after each additional point is sampled. Registration accuracy may, in examples, be a composite of two sets of measures—(i) how far each sampled point is from the bone model and (ii) a covariance indicating the uncertainty that exists in all six degrees of freedom. The error metric at any point in time may be a function of each sampled point, i.e., a composite/aggregate of the errors relative to each of those points. RMS error uses the points-to-surface distances. Accuracy may be determined after each additional point is sampled so that the registration process may be terminated as soon as the desired accuracy is achieved, i.e., without the wasted time and effort of sampling more points than are needed to provide the desired accuracy. If the error after a most recently sampled point is below a predefined threshold, then the system can inform the user that registration is complete and advance the user to a next phase in the workflow.

By way of non-limiting example, the registration error threshold could be an RMS of 0.5 mm (i.e., desired accuracy is any error less than 0.5 mm). Using the process described, registration with error less than 0.5 mm was achieved in as few as 8 to 10 samples, in some experiments.

It is of interest to determine when a user has sampled sufficient points to register the bone to the preoperative plan accurately. It is not always apparent when the user has achieved an accurate registration. The algorithms can only infer the accuracy of the registration mathematically. Direct measurement of registration accuracy is not possible because of practical clinical limitations (albeit the visual cues claimed herein do facilitate surgeon input). We may wish to capture the minimum number of points required to achieve a sufficiently accurate registration in practice. To determine when the user has sampled a sufficient number of points, and consequently, an accurate registration achieved, is of commercial interest.

Notably, the ICP error metric may not be sufficiently robust to determine when the user has achieved a reasonably accurate registration. By way of nonlimiting example, we may also investigate the selected transforms' impact on the sampled data points. The variances of the spatial positions between sampled points before and after each of the respective transforms are applied may be used to infer the accuracy of the registration. A lower variance would correspond to a more accurate registration. By way of nonlimiting example, the selected transforms may correspond to the transform with the lowest ICP error metric for each sampled point after the fourth sampled point. By way of nonlimiting example, a distribution of transforms based on combinations of four points can be evaluated for each sampled point and used as a means of selecting a suitable transform.

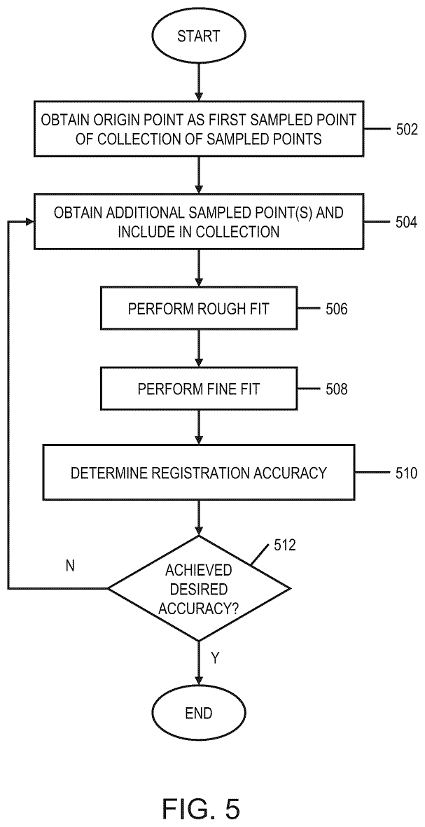

FIG. 5 depicts an example process for fitting/registering the bone model point cloud to a surface point cloud of patient anatomy, in accordance with aspects described herein. The process can be performed by a computer system executing software to perform aspects discussed herein. This computer system may be the same or a different computer system than: (i) one that stores/maintains the bone model point cloud, (ii) one that obtains sampled points of patient anatomy from the probe, and/or (iii) one that presents on one or more displays a live view of the sampling/surgical environment augmented with AR elements as described herein. In this manner, there may be one or more computer systems participating in data collection and/or processing to perform aspects described herein. In examples where more than one computer system is involved, such computer systems may be in wired and/or wireless data communication with each other, for instance over one or more networks.

The process of FIG. 5 obtains (502) the origin point as the first sampled point of the patient anatomy. This point is provided as part of a collection that is expanded as additional points are sampled. The process proceeds by obtaining (504) additional sampled point(s) and includes those in the collection. At 504, one or more additional sample points are collected. A point determined to be an outlier may be automatically rejected and optionally replaced by resampling at another point. In a specific example, the first iteration of 504 collects and adds two additional sample points to the collection so that the collection includes three points before progressing.

The process then proceeds to attempt to fit the bone model point cloud to the surface point cloud defined by the points existing in the collection at that time. The process performs (506) a rough fit (for instance by applying the RANSAC algorithm) on points of the collection. In a specific example, all points existing in the collection at that point in the process are used in this fit. The process then performs (508) a fine fit (for instance by applying an ICP algorithm) on points of the collection. In a specific example, all points existing in the collection at that point in the process are used in this fit. The process then determines (510) the registration accuracy and inquires (512) whether the desired accuracy is achieved (for instance based on one or more thresholds defining desired registration accuracy). If so (512, Y), the process ends, as the point clouds have been registered to each other with sufficient accuracy. The points of the point cloud of the bone model, once registered to the patient anatomy, can then be taken as an accurate reflection of the surface points of the patient anatomy for use in surgical activities.

If instead it is determined that the desired accuracy has not yet been achieved (512, N), the process iterates back to 504 where is obtains additional sampled point(s) to include in the collection, and proceeds again through the rough and fine fittings (506, 508) using the points then existing in the collection (which includes the additional sampled point(s)). In specific examples, only one additional sampled point is collected when iterating back to 504 from 512 before repeating the rough and fine fittings. Accordingly, in such examples, the registration accuracy and determination whether further sampling is needed is performed after each additional sample point is collected.

The presentation of the bone model in AR can provide a visualization of the real-time transform of the bone model point cloud overlaid on the actual patient anatomy, giving the user an intuitive understanding of how the registration process works and providing the user with an updating visual representation of the registration accuracy. These visual cues make registration intuitive and promote added safety. For instance, in conventional systems that require sampling of, for example, 40 points, the user's attention may be directed away from the surgical area to a display monitor. The provided AR overlay in accordance with aspects described herein enables the user to pay direct attention to the surgical area and patient anatomy while taking the relatively few number of required samples to achieve the desired accuracy. The visualization of how the bone model point cloud transform changes with each sampled point enables the user to intuitively assess the registration accuracy.

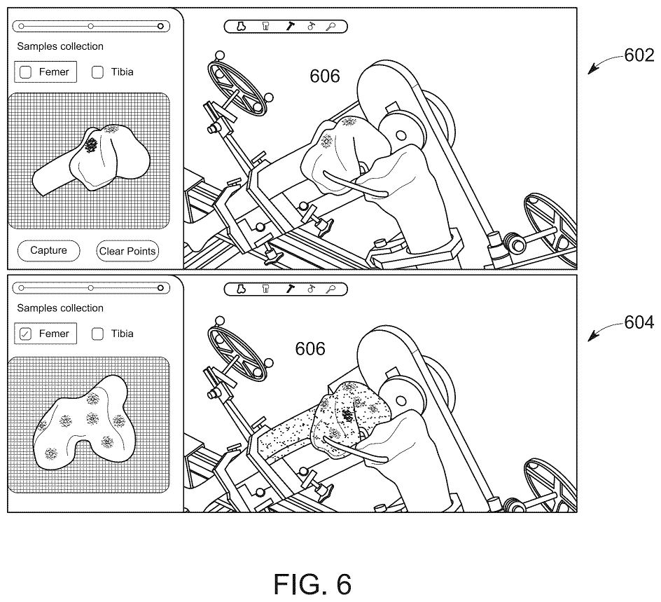

FIG. 6 shows an example (in 602) of the bone model fit after sampling two points. As the user samples additional points, the registration transform for the bone model point cloud is updated via the augmented reality overlay 606, enabling the user to watch the registration accuracy improve with each sampled point until the two point clouds are registered (in 604).

An additional limitation of existing methods, as noted above, is that the point sampling is often conducted in specific order wherein the user captures a diverse and comprehensive point cloud, albeit in a highly inefficient way. The goal is to generate a point cloud representative of the patient bone surface anatomy and solve for the transform of the pre-operative bone model (generated from the CT scan) that minimizes the error metric between these point clouds. Current systems direct the user to sample ordered bone surface points of the patient's anatomy via screen prompts represented by circles on a virtual bone model rendering of the bone model from the CT scan. The next point to be sampled may be a different color or a different diameter as a user prompt. The bone model rendering is not oriented to the actual patient position but arbitrarily positioned and free-floating. While rotatable by the surgeon, it is incumbent on the user to orient the bone model to a suitable position. This process is highly inefficient, unintuitive, and cumbersome for the user.

Aspects described herein do not constrain the user to ordered points—the user can sample any points of interest until the process determines the registration accuracy is below the acceptable threshold. Notably, the user can be prompted to capture a diverse set of points, but the position and order of those points is not a system constraint. By way of non-limiting examples, the system could display points of interest for the user to register with the probe tip that can be captured in any order. By way of non-limiting example, the system could show visual representations of points/regions already sampled by the surgeon, enabling the surgeon to visualize the areas that have not yet been sampled.

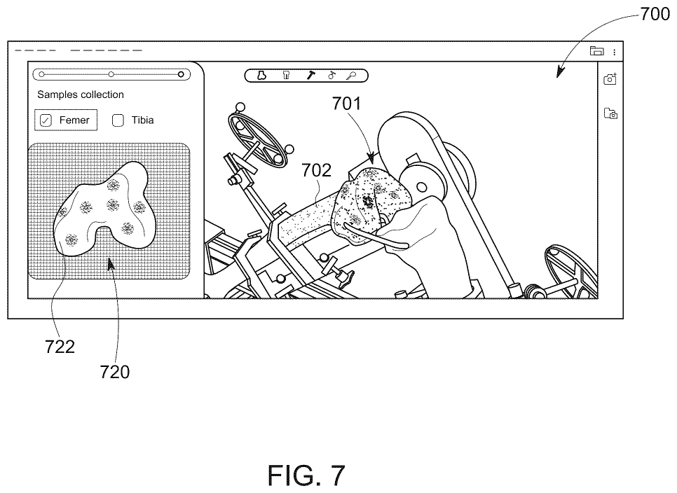

Referring to FIG. 7, a view 700 of the environment displays the bone model 702 in AR as an overlay (i.e., interposed in the user's view to the actual patient anatomy) of the bone. Points 701 on bone model 702 indicate sampled points of the patient anatomy (bone) and window 720 presents the computer-generated bone model 722 showing where the system determines those sampled points 701 to be on the bone model 722. Visual representations of regions already sampled by the surgeon can provide a visual cue of areas that have not yet been sampled. Therefore, additionally or alternatively, the system could indicate ‘points of interest’ in view 700 as suggested points for the user to sample in any order.

Example processes can also include an outlier rejection approach to overcome the limitation of collecting erroneous samples, for instance a sample in the air or other location that is not against the patent anatomy or interest. This increases the robustness of the system. The process can incorporate an auto-rejection feature to reject a sampled point on-the-fly (i.e., before sampling is concluded) if it deviates too much from the rest of the point cloud. In current approaches that sample 40 or more points, discovery of an outlier point would require that the sampling be restarted from the first point.

In some embodiments, a single tracking camera is used. This constrains the orientation of the view to one angle, but it is noted that additional tracking camera(s) could be added to the system, for instance to help orient in three dimensions more accurately. For instance, more than one tracking camera can be used to facilitate three-dimensional alignment of the initial bone model pose.

Using techniques described herein in a cadaver lab, it was demonstrated that the RMS error for a femur real-time registration on a femur was 0.40 mm with less than 10 sampled points.

Shortcomings of the prior art are overcome and additional advantages are provided through the provision of computer-implemented methods, computer systems configured to perform methods, and computer program products that include computer readable storage media storing instructions for execution to perform methods described herein. Additional features and advantages are realized through the concepts described herein.

In one example of a computer-implemented method, the method includes registering a bone model point cloud to a point cloud of patient anatomy. The registering includes obtaining a user selection of an origin point for the bone model point cloud. The origin point may be a sampled surface point on patient anatomy and may be a first point included in an established collection of sample points of the patient anatomy, the collection forming the point cloud of the patient anatomy. The registering additionally includes obtaining one or more other sampled surface points on the patient anatomy, and including the obtained one or more other sampled surface points in the collection. The registering additionally includes determining an initial pose of the bone model point cloud based on the collection of sample points of the patient anatomy, obtaining an additional sampled surface point on the patient anatomy and updating the collection of sample points to include the additional sampled surface point and thereby provide an updated collection of sample points, determining a fit of the bone model point cloud to the point cloud of the patient anatomy based on the updated collection of sample points of the patient anatomy, determining a registration accuracy of the fit of the bone model point cloud to the point cloud of the patient anatomy, and performing processing based on the determined registration accuracy.

In embodiments, the performing processing includes, based on the determined registration accuracy being less than a preconfigured threshold level of accuracy, iterating, one or more times, the obtaining an additional sampled surface point, the determining a fit, and the determining the registration accuracy. In embodiments, the iterating halts based on the determined registration accuracy being at least the preconfigured threshold level of accuracy. In embodiments, based on halting the iterating, the determined fit of the bone model point cloud to the point cloud of the patient anatomy provides a registration of the bone model point cloud to the point cloud of the patient anatomy, and the method further includes determining and digitally presenting to a surgeon one or more indications of surgical guidance.

In some embodiments, obtaining the user selection of the origin point includes providing a bone model augmented reality (AR) element overlaying a portion of a view to the patient anatomy. The view can show a registration probe, and the bone model AR element can be provided at a fixed position relative to a probe tip of the probe. User movement of the probe can reposition the bone model AR element and the user selection of the origin point can include the user positioning and orienting the bone model AR element in the view to overlay the patient anatomy by touching the patient anatomy with the probe tip, and then providing some input (e.g., a mouse click, button press, verbal confirmation, or the like) to select the origin point as a position of the probe tip touching the patient anatomy. Further, in some examples obtaining the user selection of the origin point includes providing a probe axis AR element overlaying another portion of the view to the patient anatomy. The probe axis AR element can include an axis line extending from the probe at a first position (for instance the tip) and away from the probe tip to a second position, where the axis line represent an axis of the probe/probe tip.

Additionally or alternatively, determining the fit of the bone model point cloud to the point cloud of the patient anatomy based on the updated collection of sample points of the patient anatomy can include performing a rough fitting of the bone model point cloud to the point cloud of the patient anatomy using the updated collection of sample points of the patient anatomy and, based on performing the rough fitting, performing a fine fitting of the bone model point cloud to the point cloud of the patient anatomy using the updated collection of sample points of the patient anatomy. In embodiments, performing the rough fitting includes applying a random sample consensus (RANSAC) algorithm and/or performing the fine fitting includes applying an iterative closest point (ICP) algorithm.

Additionally or alternatively, determining the initial pose of the bone model point cloud can also utilize rough-fitting and/or fine-fitting. For instance, determining the initial pose of the bone model (e.g., after the first two or three sampled points for instance) can include performing a rough fitting of the bone model point cloud to the point cloud of the patient anatomy by applying a random sample consensus (RANSAC) algorithm and, based on performing the rough fitting, performing a fine fitting of the bone model point cloud to the point cloud of the patient anatomy by applying an iterative closest point (ICP) algorithm.

It is noted that it may not be even possible, let alone practical, for a human to mentally perform the registration of two point clouds. For instance, point clouds are composed of digital representations of points in space, and applying algorithms to register two point clouds of even just two or more points each may not be practical or possible in the human mind, let alone at speeds required in surgical and other applications. Furthermore, it is not possible to sample points on patient anatomy purely mentally and obtain point data that can be used in computations to register point clouds. A bone model point cloud in accordance with aspects described herein is a digital construct and does not exist mentally. Further, it is not possible to provide augmented reality purely in the human mind, for instance to overlay digital graphical elements as AR elements over a view to an environment. In addition, point cloud registration is vitally important for surgical operative planning and execution, and the safety and success of the corresponding surgical procedures. Aspects described herein at least improve the technical fields of registration, surgical practices, and other technologies.

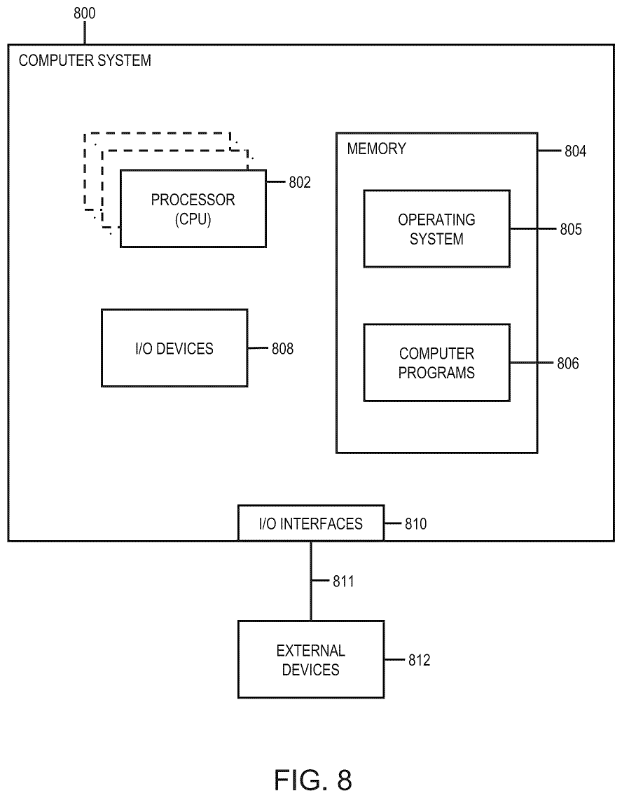

Processes described herein may be performed singly or collectively by one or more computer systems, such as one or more systems that are, or are in communication with, a registration probe, camera system, tracking system, and/or AR system, as examples. FIG. 8 depicts one example of such a computer system and associated devices to incorporate and/or use aspects described herein. A computer system may also be referred to herein as a data processing device/system, computing device/system/node, or simply a computer. The computer system may be based on one or more of various system architectures and/or instruction set architectures, such as those offered by Intel Corporation (Santa Clara, California, USA) or ARM Holdings plc (Cambridge, England, United Kingdom), as examples.

FIG. 8 shows a computer system 800 in communication with external device(s) 812. Computer system 800 includes one or more processor(s) 802, for instance central processing unit(s) (CPUs). A processor can include functional components used in the execution of instructions, such as functional components to fetch program instructions from locations such as cache or main memory, decode program instructions, and execute program instructions, access memory for instruction execution, and write results of the executed instructions. A processor 802 can also include register(s) to be used by one or more of the functional components. Computer system 800 also includes memory 804, input/output (I/O) devices 808, and I/O interfaces 810, which may be coupled to processor(s) 802 and each other via one or more buses and/or other connections. Bus connections represent one or more of any of several types of bus structures, including a memory bus or memory controller, a peripheral bus, an accelerated graphics port, and a processor or local bus using any of a variety of bus architectures. By way of example, and not limitation, such architectures include the Industry Standard Architecture (ISA), the Micro Channel Architecture (MCA), the Enhanced ISA (EISA), the Video Electronics Standards Association (VESA) local bus, and the Peripheral Component Interconnect (PCI).

Memory 804 can be or include main or system memory (e.g., Random Access Memory) used in the execution of program instructions, storage device(s) such as hard drive(s), flash media, or optical media as examples, and/or cache memory, as examples. Memory 804 can include, for instance, a cache, such as a shared cache, which may be coupled to local caches (examples include L1 cache, L2 cache, etc.) of processor(s) 802. Additionally, memory 804 may be or include at least one computer program product having a set (e.g., at least one) of program modules, instructions, code or the like that is/are configured to carry out functions of embodiments described herein when executed by one or more processors.

Memory 804 can store an operating system 805 and other computer programs 806, such as one or more computer programs/applications that execute to perform aspects described herein. Specifically, programs/applications can include computer readable program instructions that may be configured to carry out functions of embodiments of aspects described herein.

Examples of I/O devices 808 include but are not limited to microphones, speakers, Global Positioning System (GPS) devices, RGB and/or IR cameras, lights, accelerometers, gyroscopes, magnetometers, sensor devices configured to sense light, proximity, heart rate, body and/or ambient temperature, blood pressure, and/or skin resistance, registration probes and activity monitors. An I/O device may be incorporated into the computer system as shown, though in some embodiments an I/O device may be regarded as an external device (812) coupled to the computer system through one or more I/O interfaces 810.

Computer system 800 may communicate with one or more external devices 812 via one or more I/O interfaces 810. Example external devices include a keyboard, a pointing device, a display, and/or any other devices that enable a user to interact with computer system 800. Other example external devices include any device that enables computer system 800 to communicate with one or more other computing systems or peripheral devices such as a printer. A network interface/adapter is an example I/O interface that enables computer system 800 to communicate with one or more networks, such as a local area network (LAN), a general wide area network (WAN), and/or a public network (e.g., the Internet), providing communication with other computing devices or systems, storage devices, or the like. Ethernet-based (such as Wi-Fi) interfaces and Bluetooth® adapters are just examples of the currently available types of network adapters used in computer systems (BLUETOOTH is a registered trademark of Bluetooth SIG, Inc., Kirkland, Washington, U.S.A.).

The communication between I/O interfaces 810 and external devices 812 can occur across wired and/or wireless communications link(s) 811, such as Ethernet-based wired or wireless connections. Example wireless connections include cellular, Wi-Fi, Bluetooth®, proximity-based, near-field, or other types of wireless connections. More generally, communications link(s) 811 may be any appropriate wireless and/or wired communication link(s) for communicating data.

Particular external device(s) 812 may include one or more data storage devices, which may store one or more programs, one or more computer readable program instructions, and/or data, etc. Computer system 800 may include and/or be coupled to and in communication with (e.g., as an external device of the computer system) removable/non-removable, volatile/non-volatile computer system storage media. For example, it may include and/or be coupled to a non-removable, non-volatile magnetic media (typically called a “hard drive”), a magnetic disk drive for reading from and writing to a removable, non-volatile magnetic disk (e.g., a “floppy disk”), and/or an optical disk drive for reading from or writing to a removable, non-volatile optical disk, such as a CD-ROM, DVD-ROM or other optical media.

Computer system 800 may be operational with numerous other general purpose or special purpose computing system environments or configurations. Computer system 800 may take any of various forms, well-known examples of which include, but are not limited to, personal computer (PC) system(s), server computer system(s), such as messaging server(s), thin client(s), thick client(s), workstation(s), laptop(s), handheld device(s), mobile device(s)/computer(s) such as smartphone(s), tablet(s), and wearable device(s), multiprocessor system(s), microprocessor-based system(s), telephony device(s), network appliance(s) (such as edge appliance(s)), virtualization device(s), storage controller(s), set top box(es), programmable consumer electronic(s), network PC(s), minicomputer system(s), mainframe computer system(s), and distributed cloud computing environment(s) that include any of the above systems or devices, and the like.

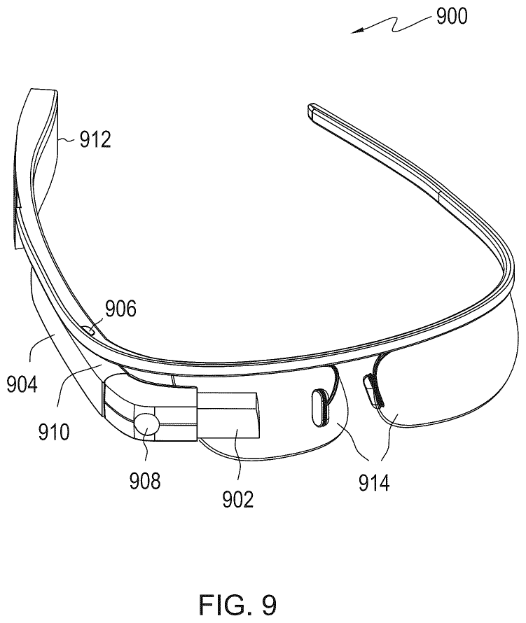

FIG. 9 depicts another example of a computer system to incorporate and use aspects described herein. FIG. 9 depicts an example eyewear based wearable device, for instance a wearable smart glasses device to facilitate presentation of AR elements to a wearer of the device. Device 900 can include many of the same types of components included in computer system 800 described above. In the example of FIG. 9, device 900 is configured to be wearable on the head of the device user. The device includes a display 902 that is positioned in a peripheral vision line of sight of the user when the device is in operative position on the user's head. Suitable displays can utilize LCD, CRT, or OLED display technologies, as examples. Lenses 914 may optionally include active translucent displays, in which an inner and/or outer surface of the lenses are capable of displaying images and other content. This provides the ability to impose this content directly into the line of sight of the user, overlaying at least part of the user's view to the environment through the lenses. In particular embodiments described herein, content presented on the lens displays are AR elements overlaying a stream from camera(s) depicting a surgical environment/theater.

Device 900 also includes touch input portion 904 that enable users to input touch-gestures in order to control functions of the device. Such gestures can be interpreted as commands, for instance a command to take a picture, or a command to launch a particular service. Device 900 also includes button 909 in order to control function(s) of the device. Example functions include locking, shutting down, or placing the device into a standby or sleep mode.

Various other input devices are provided, such as camera 608, which can be used to capture images or video. The camera can be used by the device to obtain image(s)/video of a view of the wearer's environment to use in, for instance, capturing images/videos of a scene. Additionally, camera(s) may be used to track the user's direction of eyesight and ascertain where the user is looking, and track the user's other eye activity, such as blinking or movement.

One or more microphones, proximity sensors, light sensors, accelerometers, speakers, GPS devices, and/or other input devices (not labeled) may be additionally provided, for instance within housing 910. Housing 910 can also include other electronic components, such as electronic circuitry, including processor(s), memory, and/or communications devices, such as cellular, short-range wireless (e.g., Bluetooth), or Wi-Fi circuitry for connection to remote devices. Housing 910 can further include a power source, such as a battery to power components of device 900. Additionally or alternatively, any such circuitry or battery can be included in enlarged end 912, which may be enlarged to accommodate such components. Enlarged end 912, or any other portion of device 900, can also include physical port(s) (not pictured) used to connect device 900 to a power source (to recharge a battery) and/or any other external device, such as a computer. Such physical ports can be of any standardized or proprietary type, such as Universal Serial Bus (USB).

Aspects of the present invention may be a system, a method, and/or a computer program product, any of which may be configured to perform or facilitate aspects described herein.

In some embodiments, aspects of the present invention may take the form of a computer program product, which may be embodied as computer readable medium(s). A computer readable medium may be a tangible storage device/medium having computer readable program code/instructions stored thereon. Example computer readable medium(s) include, but are not limited to, electronic, magnetic, optical, or semiconductor storage devices or systems, or any combination of the foregoing. Example embodiments of a computer readable medium include a hard drive or other mass-storage device, an electrical connection having wires, random access memory (RAM), read-only memory (ROM), erasable-programmable read-only memory such as EPROM or flash memory, an optical fiber, a portable computer disk/diskette, such as a compact disc read-only memory (CD-ROM) or Digital Versatile Disc (DVD), an optical storage device, a magnetic storage device, or any combination of the foregoing. The computer readable medium may be readable by a processor, processing unit, or the like, to obtain data (e.g., instructions) from the medium for execution. In a particular example, a computer program product is or includes one or more computer readable media that includes/stores computer readable program code to provide and facilitate one or more aspects described herein.

As noted, program instruction contained or stored in/on a computer readable medium can be obtained and executed by any of various suitable components such as a processor of a computer system to cause the computer system to behave and function in a particular manner. Such program instructions for carrying out operations to perform, achieve, or facilitate aspects described herein may be written in, or compiled from code written in, any desired programming language. In some embodiments, such programming language includes object-oriented and/or procedural programming languages such as C, C++, C#, Java, etc.

Program code can include one or more program instructions obtained for execution by one or more processors. Computer program instructions may be provided to one or more processors of, e.g., one or more computer systems, to produce a machine, such that the program instructions, when executed by the one or more processors, perform, achieve, or facilitate aspects of the present invention, such as actions or functions described in flowcharts and/or block diagrams described herein. Thus, each block, or combinations of blocks, of the flowchart illustrations and/or block diagrams depicted and described herein can be implemented, in some embodiments, by computer program instructions.

Although various embodiments are described above, these are only examples.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components and/or groups thereof.

The corresponding structures, materials, acts, and equivalents of all means or step plus function elements in the claims below, if any, are intended to include any structure, material, or act for performing the function in combination with other claimed elements as specifically claimed. The description of one or more embodiments has been presented for purposes of illustration and description, but is not intended to be exhaustive or limited to in the form disclosed. Many modifications and variations will be apparent to those of ordinary skill in the art. The embodiment was chosen and described in order to best explain various aspects and the practical application, and to enable others of ordinary skill in the art to understand various embodiments with various modifications as are suited to the particular use contemplated.

What is claimed is:

1. A computer implemented method comprising:
registering a model point cloud to a point cloud of an object, the registering comprising:
obtaining a user selection of an origin point for the model point cloud, the origin point being a sampled surface point on the object and being a first point included in an established collection of sample points of the object, the collection forming the point cloud of the object;
obtaining one or more other sampled surface points on the object and including the obtained one or more other sampled surface points in the collection;
determining an initial pose of the model point cloud based on the collection of sample points of the object;
obtaining an additional sampled surface point on the object and updating the collection of sample points to include the additional sampled surface point and thereby provide an updated collection of sample points;
determining a fit of the model point cloud to the point cloud of the object based on the updated collection of sample points of the object;
determining a registration accuracy of the fit of the model point cloud to the point cloud of the object; and
performing processing based on the determined registration accuracy.

2. The method of claim 1, wherein the model point cloud comprises an anatomy model point cloud and wherein the object comprises a patient anatomy.

3. The method of claim 2, wherein the performing processing comprises, based on the determined registration accuracy being less than a preconfigured threshold level of accuracy: iterating, one or more times, the obtaining an additional sampled surface point, the determining a fit, and the determining the registration accuracy.

4. The method of claim 3, wherein the iterating halts based on the determined registration accuracy being at least the preconfigured threshold level of accuracy.

5. The method of claim 4, wherein based on halting the iterating, the determined fit of the anatomy model point cloud to the point cloud of the patient anatomy provides a registration of the anatomy model point cloud to the point cloud of the patient anatomy, and wherein the method further comprises determining and digitally presenting to a surgeon one or more indications of surgical guidance.

6. The method of claim 2, wherein obtaining the user selection of the origin point comprises providing an anatomy model augmented reality (AR) element overlaying a portion of a view to the patient anatomy, the view showing a registration probe, and the anatomy model AR element being provided at a fixed position relative to a probe tip of the probe, wherein user movement of the probe repositions the anatomy model AR element and wherein the user selection comprises: the user positioning and orienting the anatomy model AR element in the view to overlay the patient anatomy by touching the patient anatomy with the probe tip, and providing input to select the origin point as a position of the probe tip touching the patient anatomy.

7. The method of claim 6, wherein obtaining the user selection of the origin point further comprises providing a probe axis AR element overlaying another portion of the view to the patient anatomy, the probe axis AR element comprising an axis line extending from the probe at a first position and away from the probe tip to a second position, the axis line representing an axis of the probe.

8. The method of claim 2, wherein determining the fit of the anatomy model point cloud to the point cloud of the patient anatomy based on the updated collection of sample points of the patient anatomy comprises:
performing a rough fitting of the anatomy model point cloud to the point cloud of the patient anatomy using the updated collection of sample points of the patient anatomy; and
based on performing the rough fitting, performing a fine fitting of the anatomy model point cloud to the point cloud of the patient anatomy using the updated collection of sample points of the patient anatomy.

9. The method of claim 8, wherein performing the rough fitting comprises applying a random sample consensus (RANSAC) algorithm and/or performing the fine fitting comprises applying an iterative closest point (ICP) algorithm.

10. The method of claim 2, wherein determining the initial pose of the anatomy model point cloud comprises performing a rough fitting of the anatomy model point cloud to the point cloud of the patient anatomy by applying a random sample consensus (RANSAC) algorithm and, based on performing the rough fitting, performing a fine fitting of the anatomy model point cloud to the point cloud of the patient anatomy by applying an iterative closest point (ICP) algorithm.

11. A computer system comprising:
a memory; and
a processor in communication with the memory, wherein the computer system is configured to perform a method comprising:
registering a model point cloud to a point cloud of an object, the registering comprising:
obtaining a user selection of an origin point for the model point cloud, the origin point being a sampled surface point on the object and being a first point included in an established collection of sample points of the object, the collection forming the point cloud of the object;
obtaining one or more other sampled surface points on the object and including the obtained one or more other sampled surface points in the collection;
determining an initial pose of the model point cloud based on the collection of sample points of the object;
obtaining an additional sampled surface point on the object and updating the collection of sample points to include the additional sampled surface point and thereby provide an updated collection of sample points;
determining a fit of the model point cloud to the point cloud of the object based on the updated collection of sample points of the object;
determining a registration accuracy of the fit of the model point cloud to the point cloud of the object; and
performing processing based on the determined registration accuracy.

12. The computer system of claim 11, wherein the model point cloud comprises an anatomy model point cloud and wherein the object comprises a patient anatomy.

13. The computer system of claim 12, wherein the performing processing comprises, based on the determined registration accuracy being less than a preconfigured threshold level of accuracy: iterating, one or more times, the obtaining an additional sampled surface point, the determining a fit, and the determining the registration accuracy.

14. The computer system of claim 13, wherein the iterating halts based on the determined registration accuracy being at least the preconfigured threshold level of accuracy.

15. The computer system of claim 14, wherein based on halting the iterating, the determined fit of the anatomy model point cloud to the point cloud of the patient anatomy provides a registration of the anatomy model point cloud to the point cloud of the patient anatomy, and wherein the method further comprises determining and digitally presenting to a surgeon one or more indications of surgical guidance.

16. The computer system of claim 12, wherein obtaining the user selection of the origin point comprises providing an anatomy model augmented reality (AR) element overlaying a portion of a view to the patient anatomy, the view showing a registration probe, and the anatomy model AR element being provided at a fixed position relative to a probe tip of the probe, wherein user movement of the probe repositions the anatomy model AR element and wherein the user selection comprises: the user positioning and orienting the anatomy model AR element in the view to overlay the patient anatomy by touching the patient anatomy with the probe tip, and providing input to select the origin point as a position of the probe tip touching the patient anatomy.

17. The computer system of claim 16, wherein obtaining the user selection of the origin point further comprises providing a probe axis AR element overlaying another portion of the view to the patient anatomy, the probe axis AR element comprising an axis line extending from the probe at a first position and away from the probe tip to a second position, the axis line representing an axis of the probe.

18. The computer system of claim 12, wherein determining the fit of the anatomy model point cloud to the point cloud of the patient anatomy based on the updated collection of sample points of the patient anatomy comprises:
performing a rough fitting of the anatomy model point cloud to the point cloud of the patient anatomy using the updated collection of sample points of the patient anatomy; and
based on performing the rough fitting, performing a fine fitting of the anatomy model point cloud to the point cloud of the patient anatomy using the updated collection of sample points of the patient anatomy.

19. The computer system of claim 18, wherein performing the rough fitting comprises applying a random sample consensus (RANSAC) algorithm and/or performing the fine fitting comprises applying an iterative closest point (ICP) algorithm.

20. The computer system of claim 12, wherein determining the initial pose of the anatomy model point cloud comprises performing a rough fitting of the anatomy model point cloud to the point cloud of the patient anatomy by applying a random sample consensus (RANSAC) algorithm and, based on performing the rough fitting, performing a fine fitting of the anatomy model point cloud to the point cloud of the patient anatomy by applying an iterative closest point (ICP) algorithm.

21. A computer program product comprising:
a computer readable storage medium readable by a processing circuit and storing instructions for execution by the processing circuit for performing a method comprising:
registering a model point cloud to a point cloud of an object, the registering comprising:
obtaining a user selection of an origin point for the model point cloud, the origin point being a sampled surface point on the object and being a first point included in an established collection of sample points of the object, the collection forming the point cloud of the object;
obtaining one or more other sampled surface points on the object and including the obtained one or more other sampled surface points in the collection;
determining an initial pose of the model point cloud based on the collection of sample points of the object;
obtaining an additional sampled surface point on the object and updating the collection of sample points to include the additional sampled surface point and thereby provide an updated collection of sample points;
determining a fit of the model point cloud to the point cloud of the object based on the updated collection of sample points of the object;
determining a registration accuracy of the fit of the model point cloud to the point cloud of the object; and
performing processing based on the determined registration accuracy.

22. The computer program product of claim 21, wherein the model point cloud comprises an anatomy model point cloud and wherein the object comprises a patient anatomy.

23. The computer program product of claim 22, wherein the performing processing comprises, based on the determined registration accuracy being less than a preconfigured threshold level of accuracy: iterating, one or more times, the obtaining an additional sampled surface point, the determining a fit, and the determining the registration accuracy.

24. The computer program product of claim 23, wherein the iterating halts based on the determined registration accuracy being at least the preconfigured threshold level of accuracy.

25. The computer program product of claim 24, wherein based on halting the iterating, the determined fit of the anatomy model point cloud to the point cloud of the patient anatomy provides a registration of the anatomy model point cloud to the point cloud of the patient anatomy, and wherein the method further comprises determining and digitally presenting to a surgeon one or more indications of surgical guidance.

26. The computer program product of claim 22, wherein obtaining the user selection of the origin point comprises providing an anatomy model augmented reality (AR) element overlaying a portion of a view to the patient anatomy, the view showing a registration probe, and the anatomy model AR element being provided at a fixed position relative to a probe tip of the probe, wherein user movement of the probe repositions the anatomy model AR element and wherein the user selection comprises: the user positioning and orienting the anatomy model AR element in the view to overlay the patient anatomy by touching the patient anatomy with the probe tip, and providing input to select the origin point as a position of the probe tip touching the patient anatomy.

27. The computer program product of claim 26, wherein obtaining the user selection of the origin point further comprises providing a probe axis AR element overlaying another portion of the view to the patient anatomy, the probe axis AR element comprising an axis line extending from the probe at a first position and away from the probe tip to a second position, the axis line representing an axis of the probe.

28. The computer program product of claim 22, wherein determining the fit of the anatomy model point cloud to the point cloud of the patient anatomy based on the updated collection of sample points of the patient anatomy comprises:
performing a rough fitting of the anatomy model point cloud to the point cloud of the patient anatomy using the updated collection of sample points of the patient anatomy; and
based on performing the rough fitting, performing a fine fitting of the anatomy model point cloud to the point cloud of the patient anatomy using the updated collection of sample points of the patient anatomy.

29. The computer program product of claim 28, wherein performing the rough fitting comprises applying a random sample consensus (RANSAC) algorithm and/or performing the fine fitting comprises applying an iterative closest point (ICP) algorithm.

30. The computer program product of claim 22, wherein determining the initial pose of the anatomy model point cloud comprises performing a rough fitting of the anatomy model point cloud to the point cloud of the patient anatomy by applying a random sample consensus (RANSAC) algorithm and, based on performing the rough fitting, performing a fine fitting of the anatomy model point cloud to the point cloud of the patient anatomy by applying an iterative closest point (ICP) algorithm.
